(19) United States
(12) Patent Application Publication
(10) Pub. No.: US 2023/0404640 A1
(43) Pub. Date: Dec. 21, 2023

(54) SHEATH ASSEMBLY FOR STERNAL WIRE

(71) Applicant: CircumFix Solutions, Inc., Piperton, TN (US)

(72) Inventors: Louis A. HOUFF, Piperton, TN (US); Kenneth W. RICHARDSON, Piperton, TN (US); Jacob H. CARROLL, Oakland, TN (US); Richard M. OBERT, Oakland, TN (US); Michael E. CARROLL, Oakland, TN (US)

(21) Appl. No.: 18/195,146

(22) Filed: May 9, 2023

Related U.S. Application Data
(60) Provisional application No. 63/346,577, filed on May 27, 2022.

(51) Int. Cl. A61B 17/88; A61B 17/82

(52) U.S. Cl. CPC A61B 17/8869; A61B 17/8076; A61B 17/823

(57) ABSTRACT

A sheath is provided. The sheath includes a core channel, and the core channel is configured to receive a tensioning element. The tensioning element is configured to circumscribe a bone, and the sheath is configured to be placed around a bone when the tensioning element received in the core channel circumscribes the bone. The sheath is configured to prevent direct contact between the tensioning element and the bone as the tensioning element is tensioned.

### CROSS REFERENCE TO RELATED APPLICATIONS

This application claims the benefit of U.S. Provisional Patent Application No. 63/346,577 (filed on May 27, 2022, and entitled “Sheath Assembly for Sternal Wire”), the entire contents of which are hereby incorporated herein by reference for all purposes.

### FIELD OF THE INVENTION

Embodiments of the present invention relate generally to a protective sheath covering a portion of a tensioning element such as a wire that makes direct contact with bone, preventing the tensioning element from abrading and damaging the bone surface when the tensioning element is under tension.

### BACKGROUND OF THE INVENTION

Sternotomy is a surgical procedure to access intrathoracic structures such as the heart, whereby the sternum and the attached rib appendages are separated in half along the longitudinal axis of the sternal bone with a saw. The result of sternotomy is illustrated in FIG. 1A. A cut 115 is formed in the sternum 111 along the midline of the long axis of the bone. This separates the sternum 111 and the associated rib cage in half sections left and right.

To repair the human chest wall following sternotomy, some pass a series of surgical wires in a looped fashion around the sternal halves. This is illustrated in FIG. 1B. Typically, one end of a wire 102 is fused to a needle to aid its insertion through various tissues surrounding the sternum 111. Once passed the needle is removed. The ends of wires 102 are then twisted together, reducing the size of the loops formed by the wire and compressing the wire against the sternum segments to hold them securely together in anatomical arrangement for healing.

Additional wires 102 are placed around the sternum 111 repeating the same procedure, with the wires 102 spaced to pass though similar gaps or intercostal spaces 117 between attached ribs 113. By this method, the wires 102 are generally parallel to one another, however, other wiring techniques are commonly practiced such as crossing wires analogous to a shoelace pattern. Furthermore, it is also not unusual for surgeons to place two wires side by side when concerned about poor bone quality or other patient factors such as obesity that might predispose wire to damage and abrade bone. In such instances, it is thought that adding additional wires can assist in counteracting higher biomechanical stresses, cooperating to prevent abrasion and damage to the delicate underlying sternal bone.

Due to the inherent soft nature of sternal bone, the high tension required to restrict the movement of the sternal segments and other patient factors such as high BMI, age, compromised bone health and numerous other underlying comorbidities, wire can easily abrade and damage the sternal bone surface delaying or interfering natural healing. Such abrasion and damage can lead to wire loosening, wire breakage, non-union (unhealed bone), and infection. Such complications might necessitate rehospitalization and surgical intervention to mitigate.

In most cases, wire fixation has proven to be a successful and cost effective method of repairing the cut sternum with minimal reports of infection and non-union. Complication rates (such as for infection and/or non-union) are as high as 8%. Patients that incur complications, however, endure significant pain and resolving their issues has proven difficult, time consuming, and expensive. Surgical wire is thin with minimal surface area which contributes to its propensity to abrade and damage the bone surface, and exposed wire can create a “cheese cutter effect.” Furthermore, many other commonly marketed products tend to be over engineered, complicated, and time-consuming to implant.

### BRIEF SUMMARY OF THE INVENTION

A sheath, a sheath assembly, and methods of using the same are provided. A tensioning element such as a surgical wire may be received in a sheath to form a sheath assembly. The sheath may cover some or all of the tensioning element to reduce or prevent the possibility of direct contact between the tensioning element and a bone. By doing so, the sheath may reduce or prevent the tensioning element from abrading and damaging the bone surface when the tensioning element is under tension. The sheath may also reduce the possibility of abrasion and damage to bone when nominal and peak physiologic loads are exerted on the bone. The sheath substantially increases the surface area of contact on the bone and therefore dissipates stresses in an improved manner compared to a tensioned exposed tensioning element.

Furthermore, by providing the sheath, a sheath assembly may be easily customized to fit appropriately on a given bone. The sheath may be bent in some instances to easily conform to the bone surface, allowing the tensioning element to be tightened around a bone while reducing the likelihood of direct contact between the tensioning element and the bone. Relief areas may be provided in some instances to increase the ease of bending the sheath. Furthermore, the sheath may have one or more teeth in some instances to assist in improving traction with the bone. The sheath may be quickly and easily added to a tensioning element and secured around a bone.

In an example embodiment, a sheath is provided. The sheath has a core channel that is configured to receive a tensioning element. The tensioning element is configured to circumscribe a bone, and the sheath is configured to be placed around a bone when the tensioning element received in the core channel circumscribes the bone. The sheath is configured to prevent direct contact between the tensioning element and the bone as the tensioning element is tensioned.

In some embodiments, the sheath may be configured to completely envelop the core channel. However, in some embodiments, the sheath may be configured to only partially envelop a portion of the core channel so that the core channel is partially exposed. In related embodiments, the sheath may have a non-uniform cross section. Further, the sheath may have a buckle, and the buckle may be configured to assist in guiding placement of the tensioning element in the core channel. The buckle may be configured to prevent inadvertent disengagement of the tensioning element from the sheath.

In some embodiments, the sheath may have a non-uniform cross section. In related embodiments, the sheath may have at least one relief area having less material as compared to a remaining portion of the sheath. Further, the relief area(s) may be configured to increase the ease of bending the sheath. Additionally, in some related embodiments, the sheath may contain at least one of a honeycomb pattern, a square grid pattern, round weepholes, or an asymmetrical pattern. Furthermore, in some embodiments, the relief area(s) may be configured to increase the ease of trimming the sheath to a desired length.

In some embodiments, the sheath may have a non-uniform cross section. Further, the sheath may have at least one relief area having less material as compared to a remaining portion of the sheath. The relief area(s) may be configured to permit free passage of blood and other biologic fluids to pass through to reach the fracture site and contribute to biological healing.

In some embodiments, the sheath may include malleable material, and the sheath may be configured to conform to the contour of the bone when the sheath circumscribes the bone. Additionally, in some embodiments, the sheath may have a tapered end, and the tapered end may be configured to reduce resistance as the sheath is pulled through the body.

In another example embodiment, a sheath assembly is provided. The sheath assembly includes a tensioning element and a sheath having a core channel. The core channel is configured to receive the tensioning element. The sheath is configured to be placed around and positioned against a bone, and the sheath is configured to reduce direct contact between the tensioning element and the bone.

In some embodiments, the tensioning element may include at least one bead. The bead(s) may have an increased thickness compared to a remainder of the tensioning element. The bead(s) may be configured to restrain movement of the tensioning element relative to the sheath.

In some embodiments, the tensioning element may be a metal wire, and the tensioning element may have two free ends. The tensioning element may be configured to be tensioned by twisting the two free ends of the tensioning element together, and tensioning of the tensioning element may compress the sheath against the bone. In some related embodiments, the sheath may include malleable material, and the sheath may be configured to conform to the contour of the bone when the sheath circumscribes the bone.

In some embodiments, the sheath may have a non-uniform cross section. In some related embodiments, the sheath may have at least one relief area having less material as compared to a remaining portion of the sheath, and the relief area(s) may be configured to increase the ease of bending the sheath.

In some embodiments, the sheath may have a non-uniform cross section, and the sheath may have at least one relief area having less material as compared to a remaining portion of the sheath. The relief area(s) may be configured to permit free passage of blood and other biologic fluids to pass through to reach the fracture site and contribute to biological healing.

In another example embodiment, a method is provided for applying a sheath assembly. The method includes providing a tensioning element; providing a sheath having a core channel; receiving the tensioning element in the core channel of the sheath; positioning the tensioning element and the sheath around a bone; and securing the tensioning element and the sheath relative to the bone. In some embodiments, securing the tensioning element and the sheath relative to the bone may include applying one or more spikes to the bone.

### BRIEF DESCRIPTION OF THE DRAWINGS

Having thus described the invention in general terms, reference will now be made to the accompanying drawings, which are not necessarily drawn to scale, and wherein:

FIG. 1A illustrates a sternum having a cut along the midline of the sternum, in accordance with some embodiments discussed herein;

FIG. 1B illustrates the sternum of FIG. 1A with wires being used to secure the sternum, in accordance with some embodiments discussed herein;

FIG. 2A illustrates a side view of an example sheath assembly having a tensioning element and a sheath, in accordance with some embodiments discussed herein;

FIG. 2B illustrates an enhanced side view of the sheath assembly of FIG. 2A, in accordance with some embodiments discussed herein;

FIG. 2C illustrates a further enhanced side view of the sheath assembly of FIG. 2A, in accordance with some embodiments discussed herein;

FIG. 2D illustrates an enhanced perspective view of the sheath assembly of FIG. 2A, in accordance with some embodiments discussed herein;

FIG. 2E illustrates a cross-sectional view of the sheath assembly of FIG. 2D about the line A′-A′, in accordance with some embodiments discussed herein;

FIG. 2F illustrates a cross-sectional view of an alternative sheath assembly, in accordance with some embodiments discussed herein;

FIG. 2G illustrates an enhanced, cross-sectional view of the sheath of FIG. 2F, in accordance with some embodiments discussed herein;

FIG. 3A illustrates a side view of another example sheath assembly having a tensioning element and a sheath, in accordance with some embodiments discussed herein;

FIG. 3B illustrates an enhanced side view of the sheath assembly of FIG. 3A, in accordance with some embodiments discussed herein;

FIG. 3C illustrates an enhanced perspective view of the sheath assembly of FIG. 3A, in accordance with some embodiments discussed herein;

FIG. 3D illustrates a front view of the sheath of FIG. 3A, in accordance with some embodiments discussed herein;

FIG. 3E illustrates a front view of the sheath of FIG. 3A with a tensioning element placed in the core channel of the sheath, in accordance with some embodiments discussed herein;

FIG. 3F illustrates a front view of the sheath of FIG. 3A with a tensioning element placed in the core channel of the sheath and with gauge pins placed in the secondary channels of the sheath, in accordance with some embodiments discussed herein;

FIG. 4A illustrates a side view of another example sheath assembly having a tensioning element and a sheath, in accordance with some embodiments discussed herein;

FIG. 4B illustrates an enhanced side view of the sheath assembly of FIG. 4A, in accordance with some embodiments discussed herein;

FIG. 4C illustrates an enhanced perspective view of the sheath assembly of FIG. 4A, in accordance with some embodiments discussed herein;

FIG. 4D illustrates a further enhanced side view of the sheath assembly of FIG. 4A, in accordance with some embodiments discussed herein;

FIG. 4E illustrates a front view of the sheath of FIG. 4A, in accordance with some embodiments discussed herein;

FIG. 4F illustrates a front view of the sheath of FIG. 4A with a tensioning element placed in the core channel of the sheath, in accordance with some embodiments discussed herein;

FIG. 5A illustrates a side view of another example sheath assembly having a tensioning element and a sheath, in accordance with some embodiments discussed herein;

FIG. 5B illustrates an enhanced view of the sheath assembly of FIG. 5A, in accordance with some embodiments discussed herein;

FIG. 5C illustrates an enhanced perspective view of the sheath assembly of FIG. 5A, in accordance with some embodiments discussed herein;

FIG. 6A illustrates a side view of another example sheath assembly having a tensioning element and a sheath, in accordance with some embodiments discussed herein;

FIG. 6B illustrates an enhanced view of the sheath assembly of FIG. 6A, in accordance with some embodiments discussed herein;

FIG. 6C illustrates a further enhanced view of the sheath assembly of FIG. 6A, in accordance with some embodiments discussed herein;

FIG. 6D illustrates an enhanced perspective view of the sheath assembly of FIG. 6A, in accordance with some embodiments discussed herein;

FIG. 6E illustrates a further enhanced perspective view of the sheath assembly of FIG. 6D, in accordance with some embodiments discussed herein;

FIG. 7A illustrates a side view of another example sheath assembly having a tensioning element and a sheath, in accordance with some embodiments discussed herein;

FIG. 7B illustrates an enhanced side view of the sheath assembly of FIG. 7A, in accordance with some embodiments discussed herein;

FIG. 7C illustrates an enhanced bottom view of the sheath assembly of FIG. 7A, in accordance with some embodiments discussed herein;

FIG. 7D illustrates an enhanced perspective view of the sheath assembly of FIG. 7A, in accordance with some embodiments discussed herein;

FIG. 8A illustrates a side view of another example sheath assembly having a tensioning element and a sheath, in accordance with some embodiments discussed herein;

FIG. 8B illustrates an enhanced side view of the sheath assembly of FIG. 8A, in accordance with some embodiments discussed herein;

FIG. 8C illustrates an enhanced perspective view of the sheath assembly of FIG. 8A where a spike may be seen, in accordance with some embodiments discussed herein;

FIG. 8D illustrates a cross-sectional view of the sheath of FIG. 8C about the line B′-B′, in accordance with some embodiments discussed herein;

FIG. 9A illustrates a side view of another example sheath assembly having a double tensioning element with sheaths on the double tensioning element, in accordance with some embodiments discussed herein;

FIG. 9B illustrates an enhanced side view of the sheath assembly of FIG. 9A, in accordance with some embodiments discussed herein; and

FIG. 10 illustrates an example method for applying a sheath assembly to secure a bone, in accordance with some embodiments discussed herein.

### DETAILED DESCRIPTION

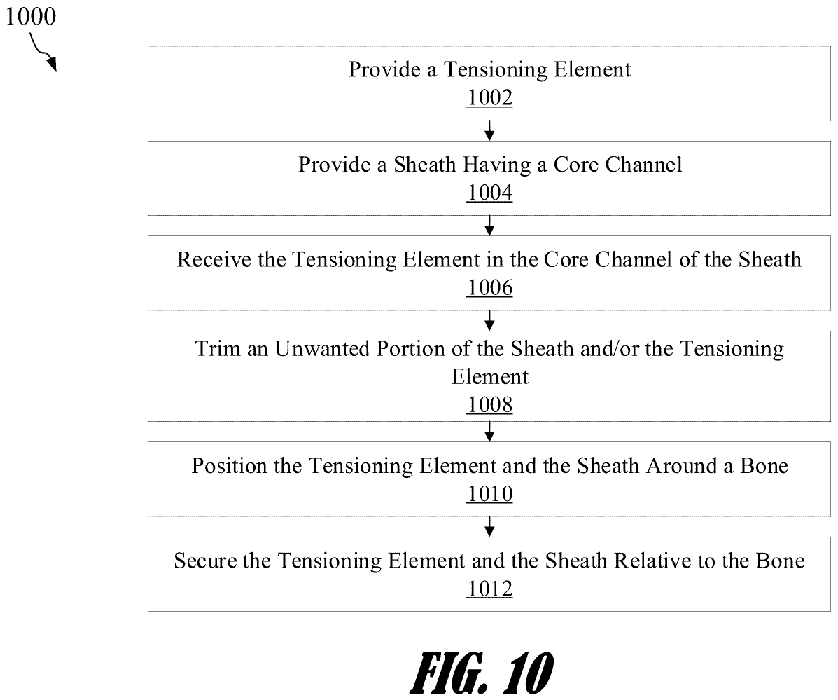

Example embodiments of the present invention now will be described more fully hereinafter with reference to the accompanying drawings, in which some, but not all embodiments of the invention are shown. Indeed, the invention may be embodied in many different forms and should not be construed as limited to the example embodiments set forth herein; rather, these embodiments are provided so that this disclosure will satisfy applicable legal requirements. Like reference numerals generally refer to like elements throughout (with the exception of elements shown in FIG. 10). For example, elements 202, 302, 402, etc. refer to a tensioning element, and elements 204, 304, 404, etc. refer to a sheath. Additionally, any connections or attachments may be direct or indirect connections or attachments unless specifically noted otherwise.

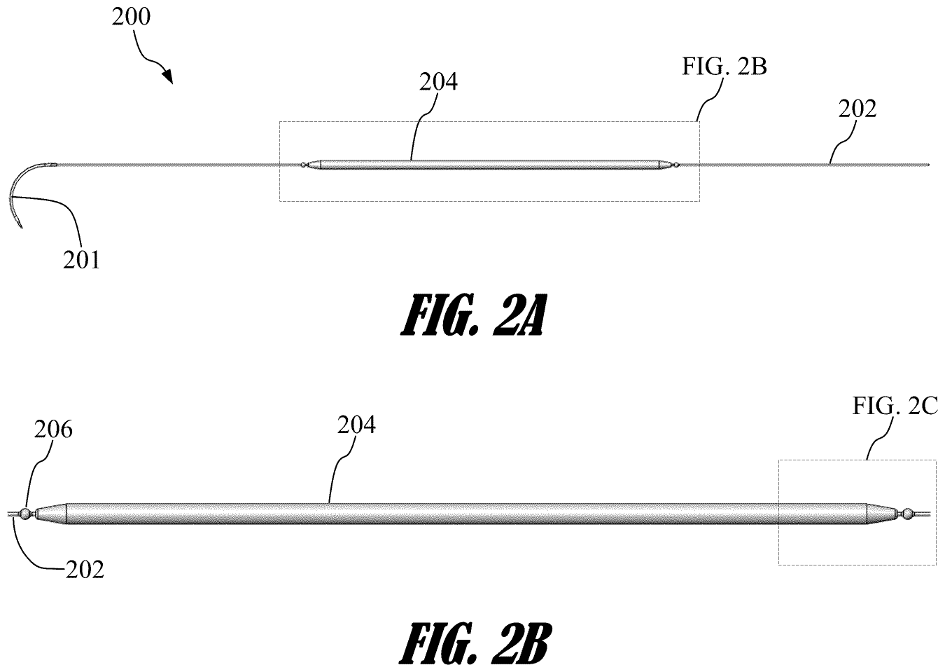

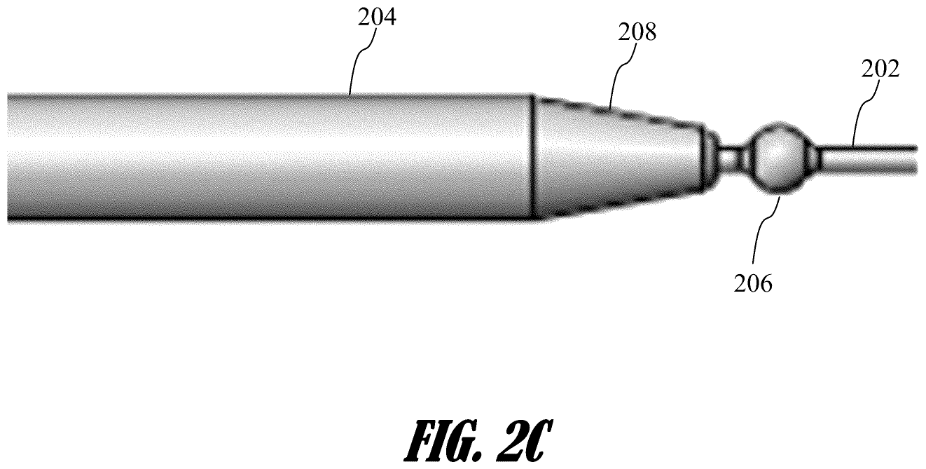

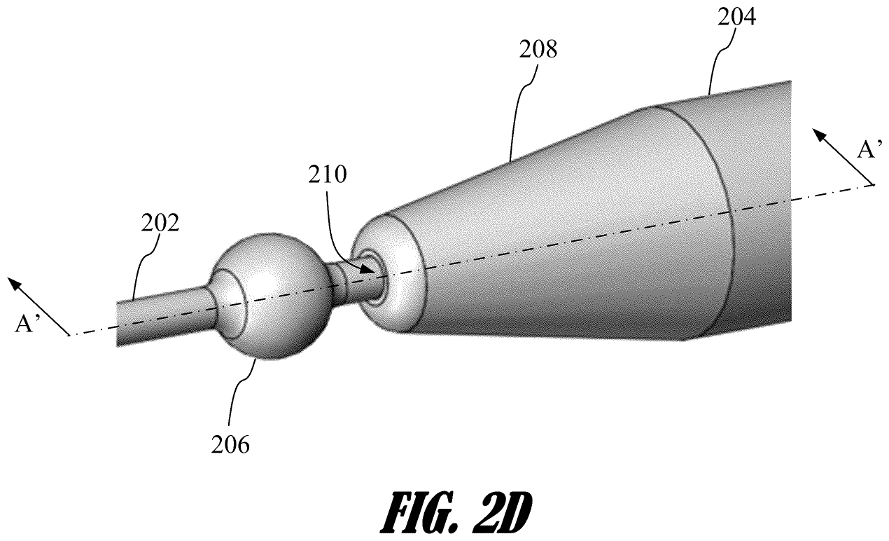

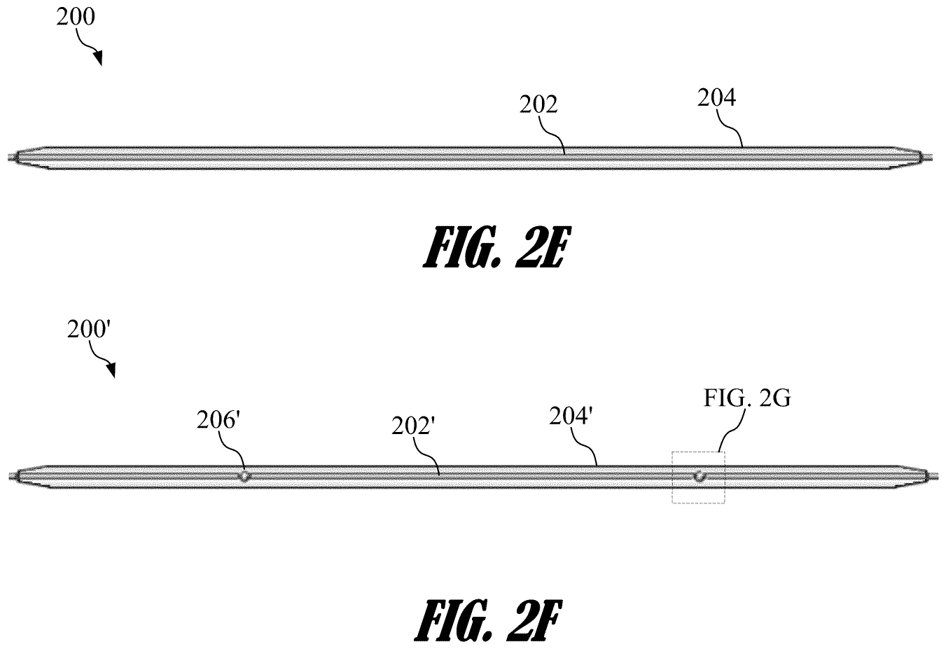

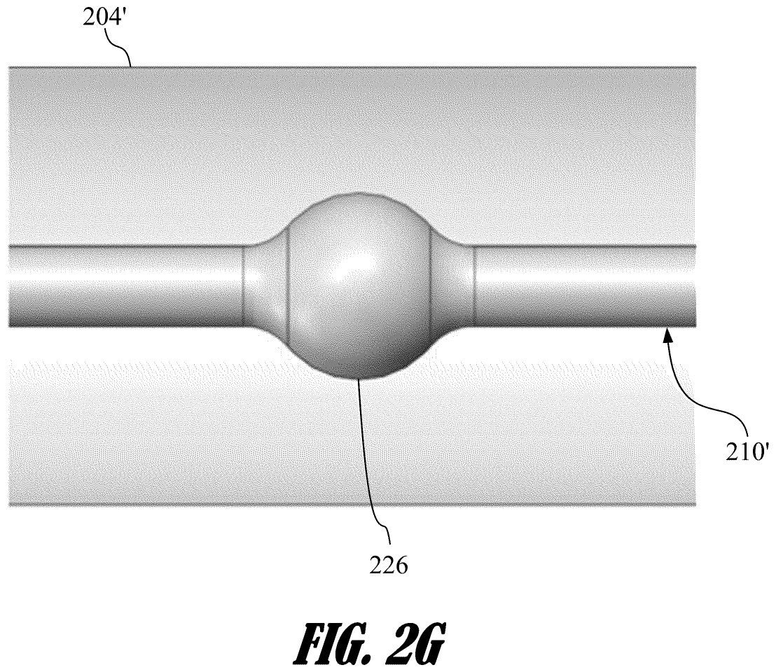

An example of one sheath assembly is illustrated in FIGS. 2A-2E. FIG. 2A illustrates a side view of an example sheath assembly 200 having a tensioning element 202 and a sheath 204, and FIG. 2B illustrates an enhanced side view of the sheath assembly 200 of FIG. 2A where the sheath 204 may be more easily seen. Additionally, FIG. 2C illustrates a further enhanced side view of the sheath assembly 200 of FIG. 2A, FIG. 2D illustrates an enhanced perspective view of the sheath assembly 200 of FIG. 2A, and FIG. 2E illustrates a cross-sectional view of the sheath assembly 200 of FIG. 2D about the line A′-A′.

As illustrated in FIG. 2A, the sheath 204 may cover a portion of or the entirety of a tensioning element 202. The sheath 204 may do so by providing a core channel and receiving the tensioning element 202 in the core channel. Thus, the sheath 204 may prevent direct contact between the tensioning element 202 and the bone surface when the sheath 204 and tensioning element 202 are placed around and positioned against a bone.

The tensioning element 202 may be a metal wire in some embodiments. Metal wire is relatively inexpensive, efficient and efficacious in most instances when used to close the chest wall following sternotomy. Where the tensioning element 202 is a metal wire, tensioning of a wire covered by a sheath through joining and twisting of its opposing ends may cause the wire and sheath 204 to deform to meet the bone surface. The tension also compresses the wire against the sheath 204, preventing the wire from separating or moving away from the sheath 204 so their relative position and contact to one another is maintained. Where a sheath 204 is used in conjunction with such a wire, this combination may maintain the benefits of using a metal wire while reducing the risk of abrasion from the wire and other risks.

The tensioning element 202 may come in other forms as well. In some embodiments, the tensioning element 202 may be a cable such as a multi-filament cable. Such multi-filament cables may be used in orthopedic applications and may also be used for repairing a severed sternum. Where such a cable is used as the tensioning element 202, the cable may be tensioned and united using tensioning instruments, crimping instruments, and/or crimping sleeves. However, the tensioning element 202 may take other forms. For example, the tensioning element 202 may be a monofilament cable, a cable tie, a strap fastener, another fastener, etc., and the sheath 204 may be configured to fully or partially envelop the tensioning element 202. The tensioning element 202 may comprise a wide variety of materials, including but not limited to stainless steel, titanium, nitonol, resorbable polymeric material, and/or non-resorbable polymeric material. In some embodiments, the tensioning element 202 may include polyether ether ketone (PEEK), polyurethane, nylon, polyethylene, silicone, plastic material, ceramic materials, or other materials. The tensioning element 202 may be formed through thermoforming, 3D printing, or other approaches.

The sheath 204 may have a variety of shapes having different lengths, widths, and cross-sectional shapes. For example, the sheath 204 may have a shape that mimics the shape/form of the underlying tensioning element 202. For example, the sheath 204 may include of a body that is round/cylindrical in shape. However, the sheath 204 may have other shapes. For example, the sheath 204 may be quadrangular (square or rectangular) with or without radiused contours to minimize soft and hard tissue irritation. Alternatively, the sheath 204 may be oval, parabolic, hexagonal or asymmetrical in shape. In one example, the sheath 204 may the central portion of the tensioning element 202 over a span to be in direct contact with bone providing a wider footprint on the bone contacting aspect of the sheath.

As illustrated in FIGS. 2B-2E, the tensioning element 202 may include a bead 206 in some embodiments. In the illustrated embodiment, the tensioning element 202 includes two beads 206, with one bead 206 on the left side of the sheath 204 and the other bead 206 on the right side of the sheath 204. Any number of beads 206 may be provided on the tensioning element 202. The beads 206 may have an increased thickness compared to a remainder of the tensioning element 202, and the beads 206 may be configured to restrain movement of the tensioning element 202 relative to the sheath 204. For example, in the illustrated embodiment in FIG. 2C, the bead 206 may restrain movement of the tensioning element 202 by preventing the tensioning element 202 from being urged further into a core channel 210 of the sheath 204.

As illustrated in FIG. 2C, the sheath 204 may include a tapered end 208. The shape and size of the taper may vary between various embodiments. The tapered end 208 of FIG. 2C includes a gradual taper. The tapered end 208 may aid the placement of the sheath 204 around a bone, reducing drag and allowing the sheath 204 to more easily pass though or around cartilage, muscle, and other tissues covering or surrounding a bone.

As illustrated in FIG. 2D, the sheath 204 may include a core channel 210, and the sheath 204 may be configured to receive the tensioning element 202 in the core channel 210. In some embodiments, the core channel 210 may be loose fitting around a tensioning element 202, allowing the tensioning element 202 and sheath 204 to move independently from one another. Conversely, in other embodiments, a sheath 204 may be configured to fit snug with the tensioning element 202, or the sheath 204 and the tensioning element 202 may be fused together to prevent the free movement of sheath 204 and tensioning element 202 from one another.

The core channel 210 of the sheath 204 illustrated in FIGS. 2A-2E may be provided such that the core channel 210 is completely enveloped. This may be advantageous to minimize direct contact between the tensioning element 202 and the bone. Further, a completely enveloped core channel 210 may reduce the likelihood of any inadvertent separation of the sheath 204 and the tensioning element 202.

The sheath 204 may be provided with varying predetermined lengths in some embodiments, and a sheath 204 having the desired length may be selected based on the size and shape of a bone and intended application. By providing varying predetermined lengths, an appropriately sized sheath 204 may be selected to minimize the amount of excess sheath material.

The sheath 204 and the tensioning element 202 may be contoured in a loop around bones having differing shapes. For example, the sheath 204 and the tensioning element 202 may be used to loop around different bones in a person's body or an animal's body, and the shape of a particular bone (e.g. the sternum) may differ from person to person. Thus, the ability to customize the fit of the sheath 204 and the tensioning element 202 may be desirable to optimize the comfort level, to ensure proper recovery, to minimize any pain, and to minimize the risk of infection or further injury.

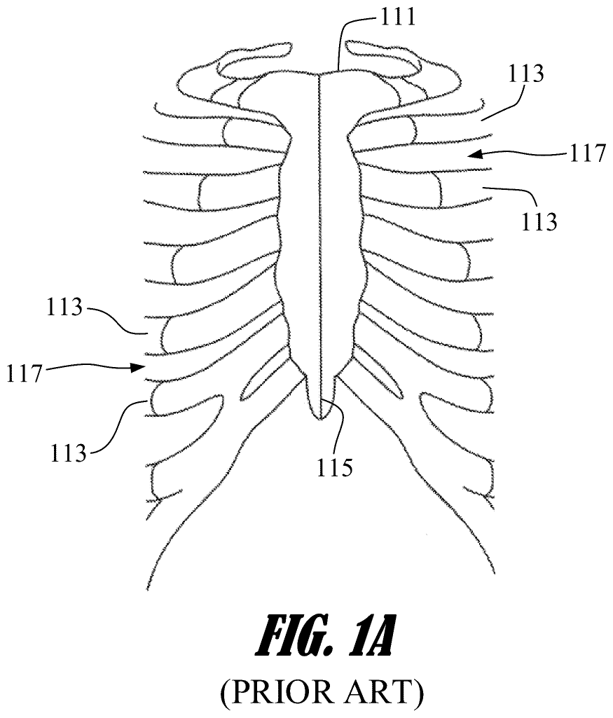

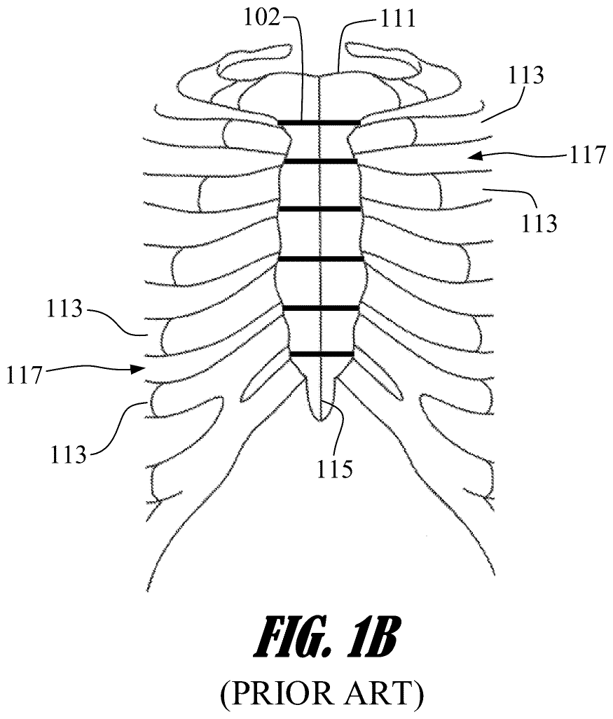

A sheath assembly 200 may be introduced into the body of a patient and passed around a bone, traveling together simultaneously in a fixed relationship to one another. For example, in sternal fixation, a sheath assembly 200 may be passed around the sternum 111 (see FIG. 1B) starting at a lateral edge in reference to the anterior facing aspect of the sternum 111 in an intercostal space 117 between attached ribs 113. The sheath assembly 200 may move posterior, or behind the sternum 111, and may emerge though a corresponding intercostal space 117 between the attached ribs 113 along the opposing lateral edge. The exposed wire ends may be united and tensioned by twisted together, forming a loop around the bone. A twisted knot may be formed typically against the anterior aspect or forward-facing surface of the sternum 111. Twisting is done until a desired amount of tension is achieved to bring the separated bone segments together in anatomical position and held and compressed together for healing. Relative movement between the reunited bone segments may be eliminated, associated pain may be reduced, and reliable healing may be accomplished.

The sheath 204 may comprise a variety of materials. The sheath 204 may include any FDA approved implant material in some embodiments. For example, the sheath 204 may comprise polyether ether ketone (PEEK), polyurethane, nylon, polyethylene, silicone, plastic material, ceramic materials, or other materials. In some embodiments, the sheath 204 may include a polymeric based material. The sheath 204 may be flexible and contourable, and this may allow it to conform to the surface of a bone along with a tensioning element 202 passing through a dedicated channel contained within its core channel 210 along its long axis. The sheath 204 may be formed through thermoforming, 3D printing, or using any other method known in the art.

Where a sheath covers a tensioning element 202 from end to end as does the sheath 204, the sheath 204 may be trimmed to expose the tensioning element 202 so that two ends of the tensioning element 202 may be physically joined together. The two ends of the tensioning element 202 may be twisted together by hand or with the assistance of an instrument. Furthermore, the tensioning element 202 may include a needle 201, and the needle 201 may be used to insert the tensioning element 202 through or around cartilage, muscle and other tissues surrounding a bone. The sheath 204 has a uniform cross section that is consistent along the length of the sheath 204, and this cross section is generally circular in shape. However, other cross sectional shapes may be used, and cross sectional shapes may be non-uniform along the length of the sheath 204.

In some embodiments, a sheath assembly may be provided having a sheath that is configured to receive beads of a tensioning element within the core channel of the sheath. FIG. 2F illustrates a cross-sectional view of such an alternative sheath assembly 200′. The sheath assembly 200′ may be provided having a sheath 204′ that is configured to receive beads 206′ of a tensioning element 202′ within the core channel of the sheath 204′. This may assist in further constraining the movement of the tensioning element 202′ relative to the sheath 204′ as the beads 206′ may generate friction or otherwise restrict the movement of the sheath 204′. Beads 206, 206′ may take any shape (e.g. disks, spherical balls, etc.).

FIG. 2G illustrates an enhanced, cross-sectional view of the sheath 204′ of FIG. 2F. The sheath 204′ may include a core channel 210′, and the core channel 210′ may have one or more enlarged portions 226 therein. The enlarged portions 226 may be configured to receive beads 206′ to further assist in restraining the movement of the tensioning element relative to the sheath 204′. In the case of a loose-fitting sheath, the tensioning element and sheath could rotate independently from each other, but the beads 206′ may generally prevent the movement of the tensioning element and the sheath end to end from each other. Beads 206′ may generally prevent slippage of the tensioning element 202′ and sheath 204′ especially where the sheath is directly molded over the tensioning element. However, in some embodiments, the beads 206′ may be urged through the core channel 210′ upon the application of a large amount of force so that the beads may be positioned in the enlarged portions 226 of the sheath 204′—where this is the case, the walls of the core channel 210′ may resist movement of the beads 206′ so that the tensioning element 202′ may not move upon the application of only small amounts of force, and this may prevent the tensioning element 202′ from being unintentionally moved.

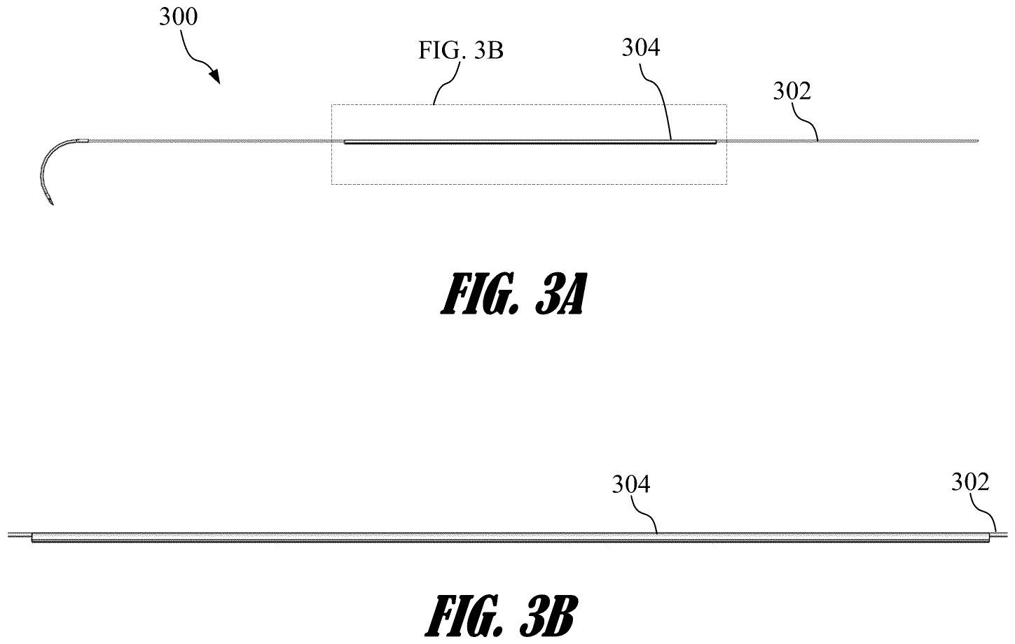

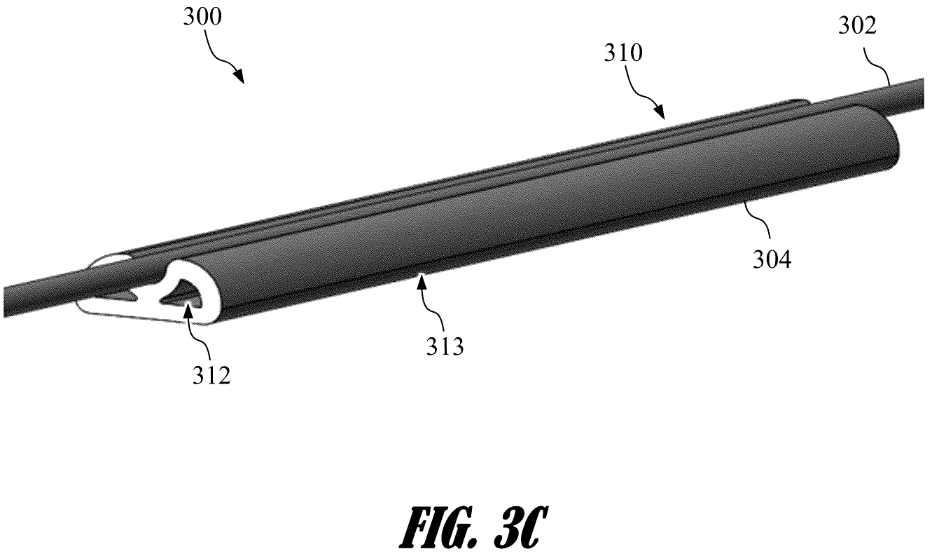

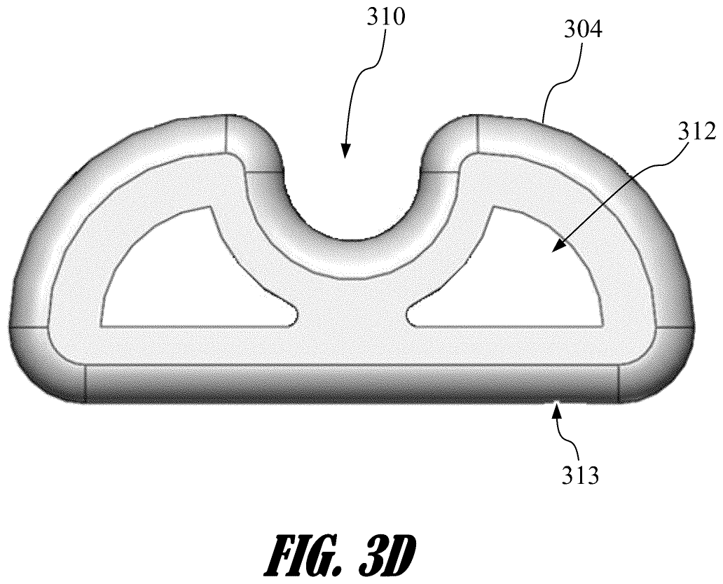

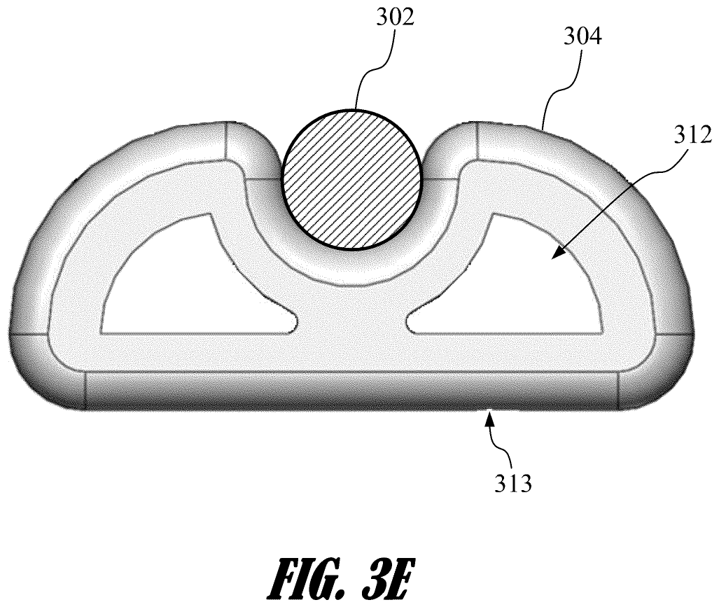

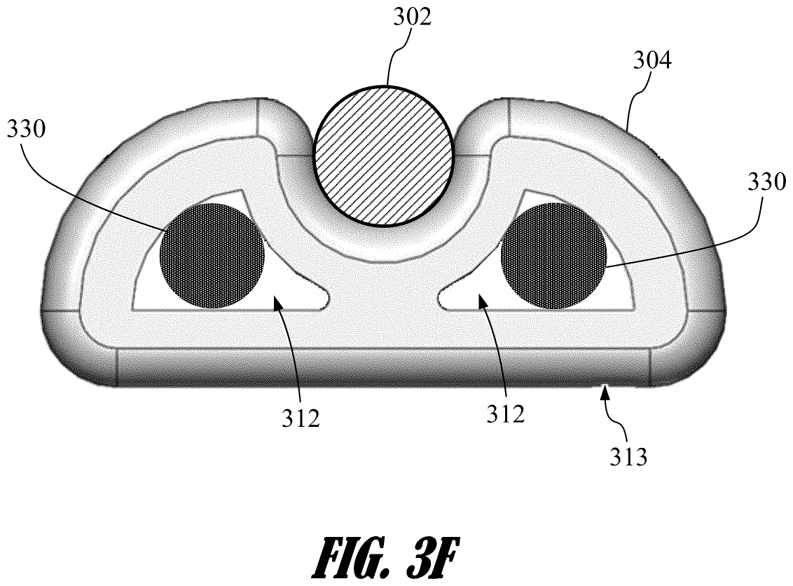

In some embodiments, a sheath may be provided having an exposed channel along its long axis where a tensioning element could rest, as opposed to a tubular configuration in which the entirely of a tensioning element is encased. FIGS. 3A-3F illustrate examples of such a sheath. FIG. 3A illustrates a side view of the example sheath assembly 300 having a tensioning element 302 and a sheath 304, FIG. 3B illustrates an enhanced side view of the sheath assembly 300 of FIG. 3A, and FIG. 3C illustrates an enhanced perspective view of the sheath assembly 300 of FIG. 3A. Furthermore, FIG. 3D illustrates a front view of the sheath 304 of FIG. 3A, and FIG. 3E illustrates a front view of the sheath 304 of FIG. 3A with a tensioning element 302 placed in the core channel 310 of the sheath 304. FIG. 3F illustrates a front view of the sheath 304 of FIG. 3A with a tensioning element 302 placed in the core channel 310 of the sheath 304 and with gauge pins 330 placed in the secondary channels 312 of the sheath 304.

Looking first at FIG. 3C, features of the sheath assembly 300 may be seen. The sheath assembly 300 may include the tensioning element 302 and the sheath 304. Further, the sheath 304 may include a core channel 310. While the core channel 210 of the sheath 204 illustrated in FIGS. 2A-2E may be provided such that the core channel 210 is completely enveloped, the sheath 304 of FIGS. 3A-3F may be configured to only partially envelop a portion of the core channel 310 so that the core channel 310 is partially exposed. The exposed portion of the core channel 310 may be directed away from any bone when the sheath assembly 300 is applied, and the bottom surface 313 of the sheath 304 may be placed in contact with the bone. The inclusion of an exposed portion may make the sheath 304 more easily deformable to meet the desired shape, and the inclusion of an exposed portion may make is easier for operators to install of a tensioning element 302 into the sheath 304.

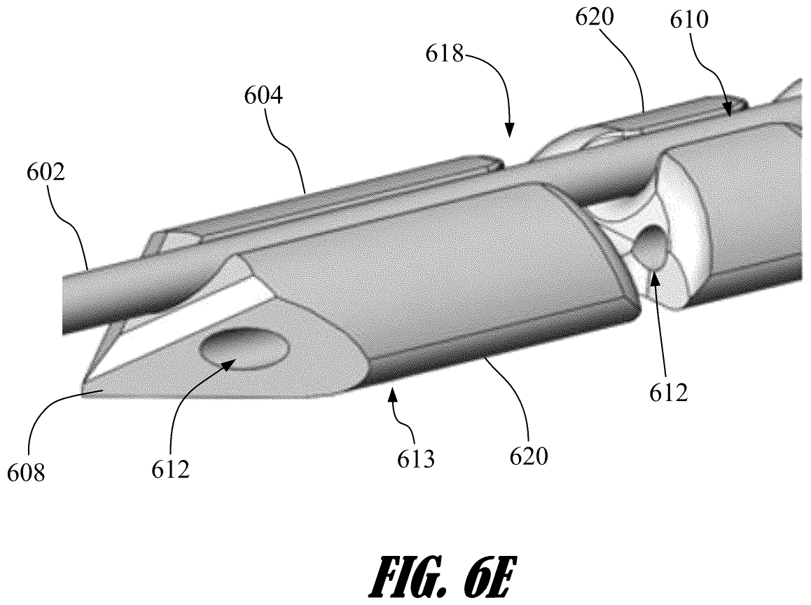

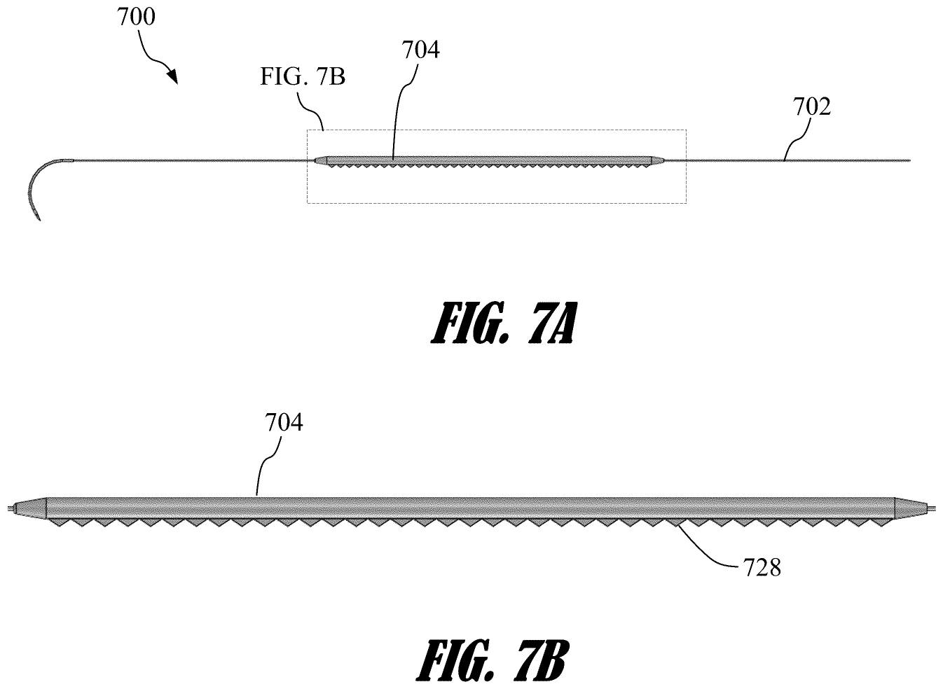

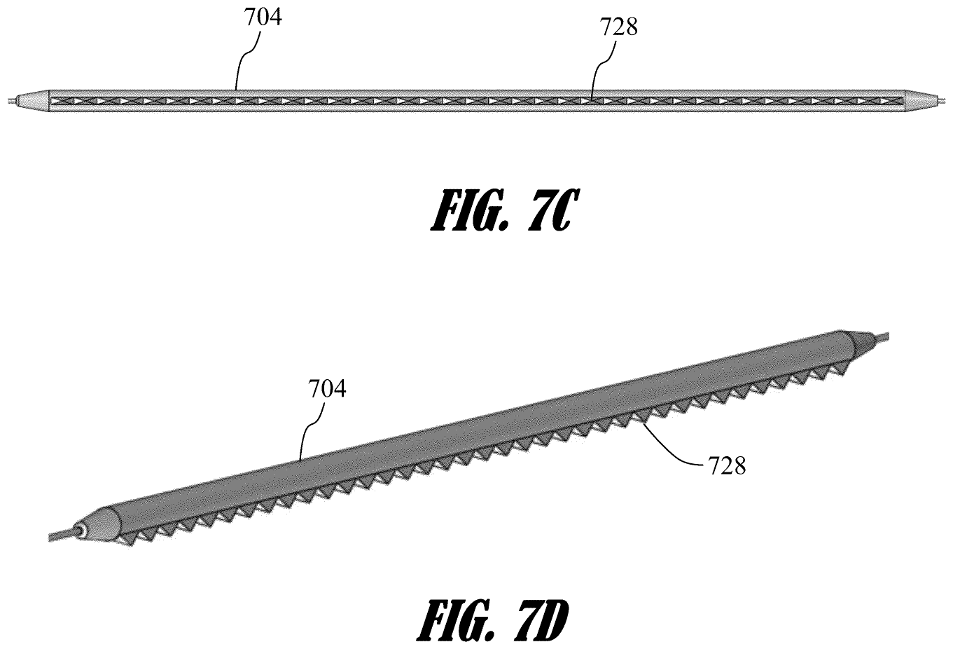

Furthermore, providing a bottom surface 313 that is flat may be beneficial to retain the sheath 304 in position, but the bottom surface 313 may have a concave or convex surface in some embodiments. As noted herein, the bottom surface 313 of the sheath 304 may have one or more spiked teeth 728 (see FIG. 7A) or a spike 828A (see FIG. 7B) to further assist in restraining the movement of the sheath 304 relative to a bone. However, other fasteners may be used as an alternative for restraining the movement of the sheath 304 such as a screw, a pin, etc. Alternatively, the sheath 304 may be provided without any such fasteners.

Turning now to FIG. 3F, a sheath 304 is shown with gauge pins 330 placed in the secondary channels 312 of the sheath 304. Gauge pins 330 may assist in increasing the rigidity of the sheath 304 where this is desired. Gauge pins 330 may run for the entire length of the sheath 304 in some embodiments, or the gauge pins 330 may be provided in smaller incremental sizes. Additional tensioning elements may be provided in the secondary channels 312 instead of gauge pins 330 in some embodiments.

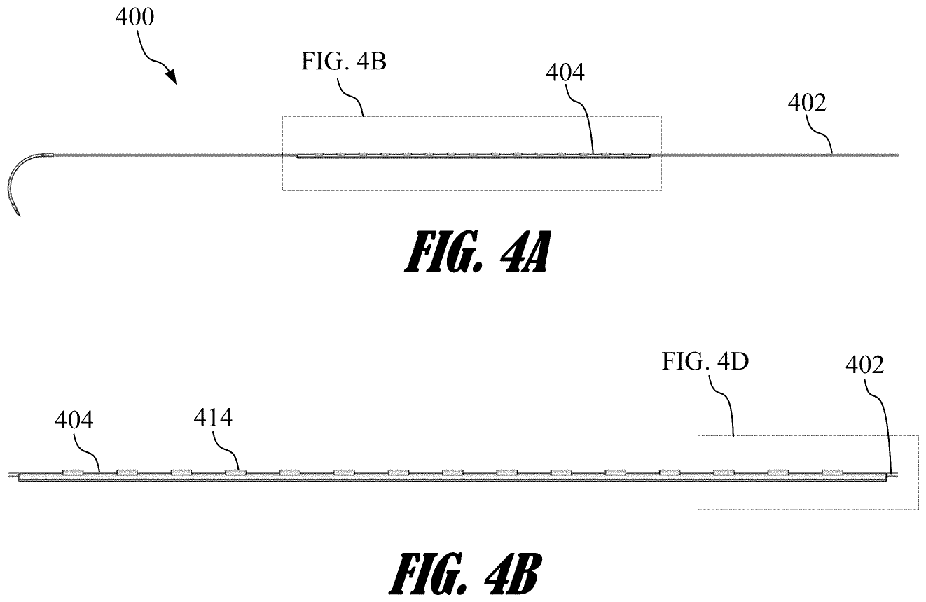

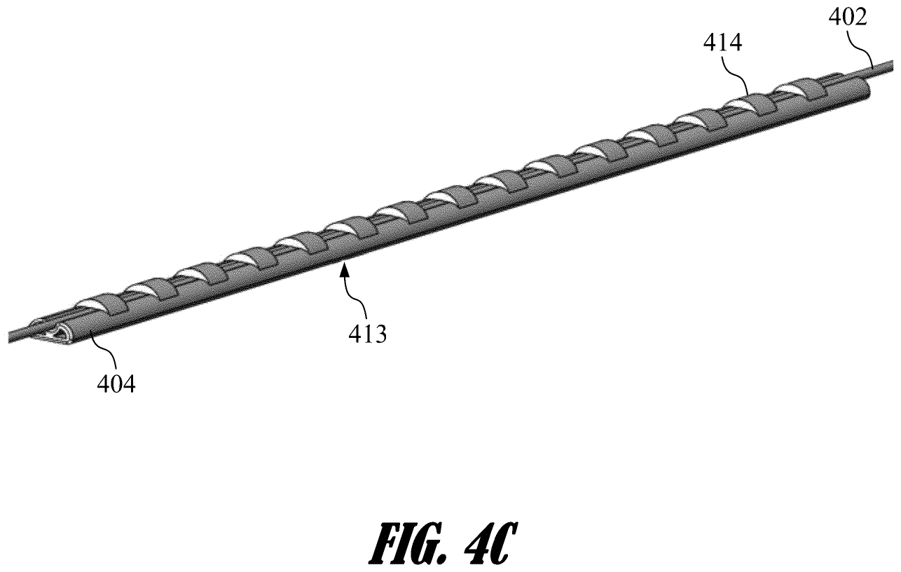

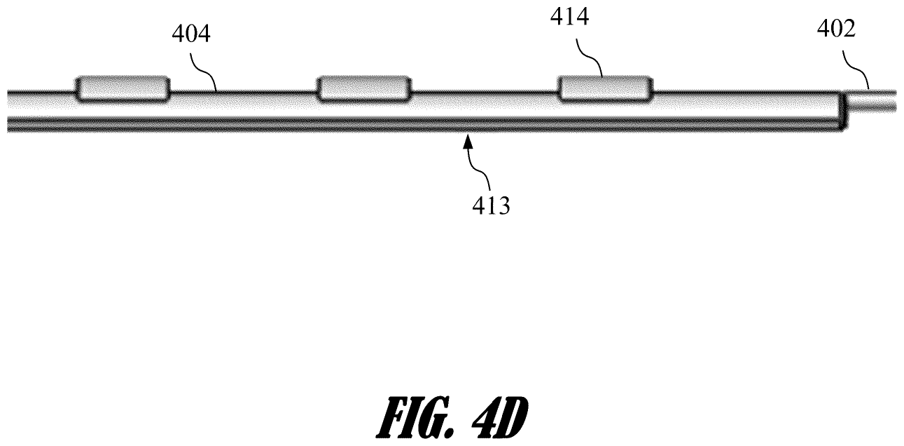

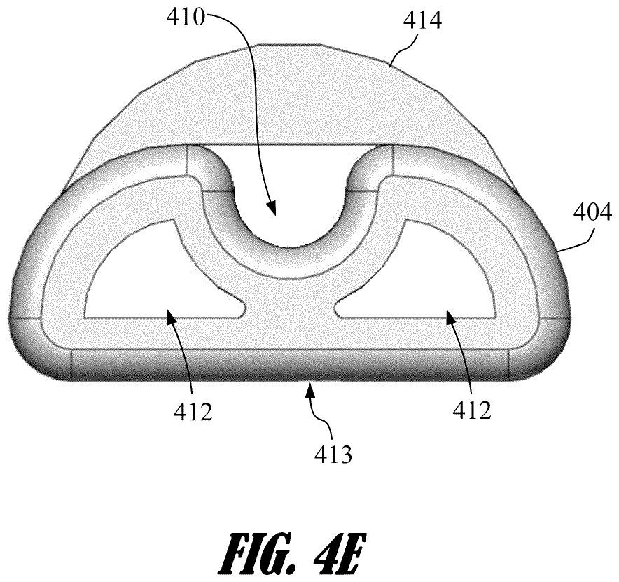

Looking now a FIG. 3C-3F, an enhanced perspective view of the sheath assembly 300 of FIG. 3A is illustrated where two secondary channels 312 may be seen. These secondary chambers 312 may extend along the long axis of the sheath 304 (e.g. the longitudinal axis of the tensioning element 302 in FIG. 3C). These secondary chambers 312 may aid in the deformation of the sheath 304, allowing the sheath 304 to bend out of plane more easily and to conform to a bone surface more easily, especially when the sheath 304 is to be looped around a cross section of a bone.

Where the sheath contains a core channel that is exposed, the channel may have coverage over the tensioning element at periodic points along its long axis. For example, buckles may be provided to assist in guiding the placement of a tensioning element and to help prevent inadvertent disengagement of the tensioning element and the sheath. FIG. 4A illustrates a side view of another example sheath assembly 400 having a tensioning element 402 and a sheath 404 having buckles 414, and the buckles 414 may be more clearly seen in the enhanced views of FIG. 4B-4D. A tensioning element 402 may be placed within the core channel of the sheath 404 beneath the buckles 414, and the buckles 414 may further restrain the tensioning element 402 relative to the sheath 404. Where an operator wishes to trim the sheath 404, the operator may do so in relief areas between the buckles 414 as these areas may have a reduced thickness relative to the areas having buckles 414.

FIGS. 4E and 4F illustrate other features of the sheath 404. In FIG. 4E, the sheath 404 is shown in isolation without any tensioning element 402. In FIG. 4F, a tensioning element 402 is provided in the core channel 410 of the sheath 404. As illustrated in FIGS. 4E and 4F, secondary channels 412 may be provided to aid in the deformation of the sheath 404, allowing the sheath 404 to bend out of plane more easily and to conform to a bone surface more easily. Further, the sheath 404 may be provided with a bottom surface 413 that may be placed in contact with a bone, and the bottom surface 413 is generally flat in the sheath 404 illustrated in FIGS. 4A-4F.

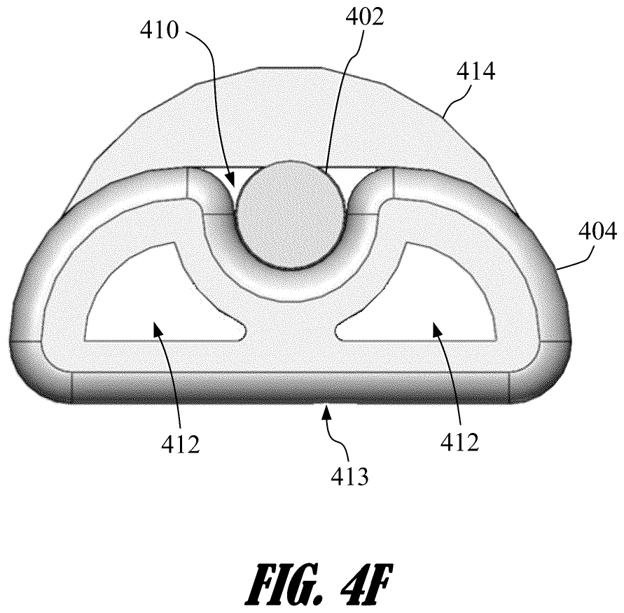

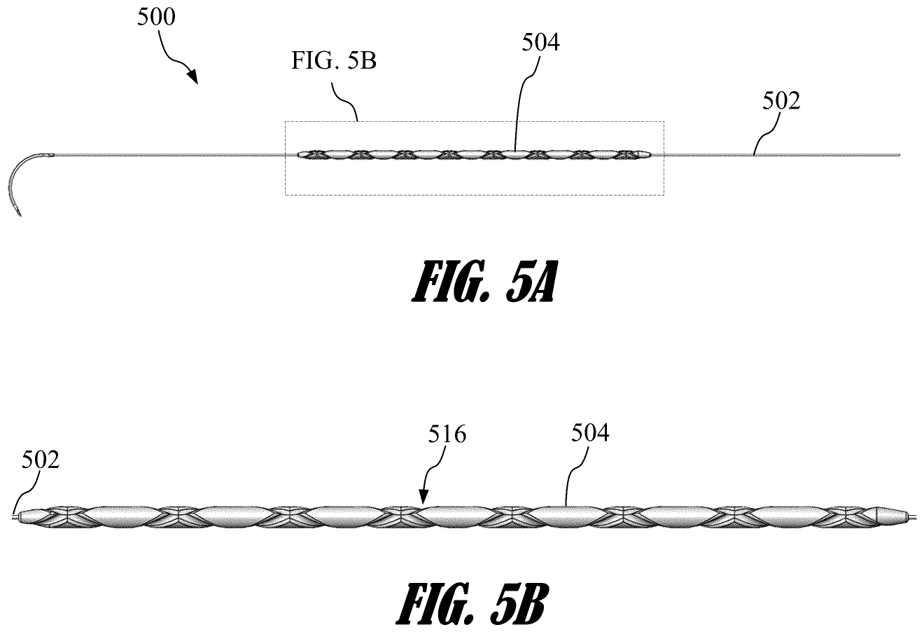

In other embodiments, relief areas may be provided in a sheath along the length of the sheath, and the relief areas may aid in the deformation of the sheath to enable the sheath to better conform to the surface of the bone. FIGS. 5A-5C illustrate one example sheath assembly 500 where the sheath 504 has relief areas in the form of pores 516. The pores 516 may be spaced throughout the surface of the sheath 504. In the embodiment illustrated in FIG. 5C, pores 516 extend throughout the full thickness of the sheath 504 and around the full surface area of the sheath 504 including any bone contacting surface. However, in other embodiments, the pores 516 may be provided only at certain areas on the sheath 504 for example, the pores 516 may not be provided at surfaces that are configured to contact a bone. Similar to other embodiments, the tensioning element 502 may be retained in a core channel of the sheath 504. Pores 516 may allow the free passage of blood and other biologic fluids to pass, allowing them to reach a fracture site at a bone and allowing them to contribute to biological healing.

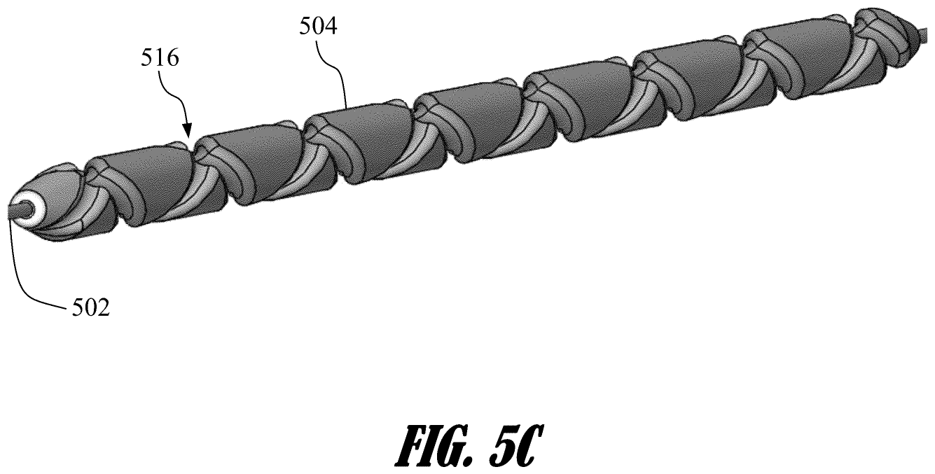

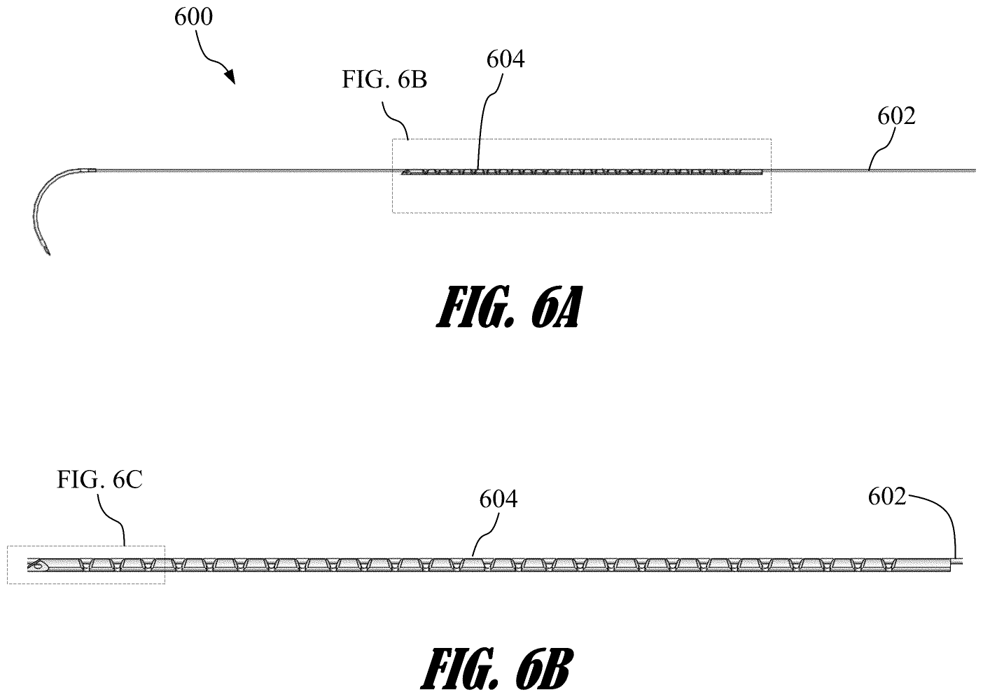

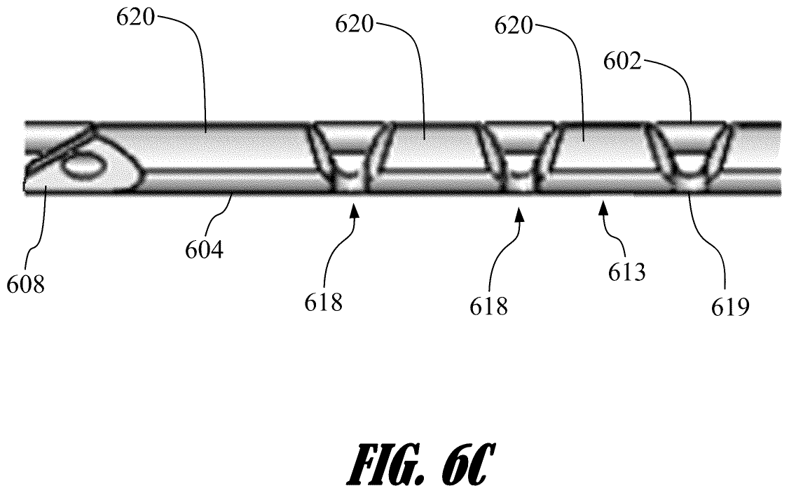

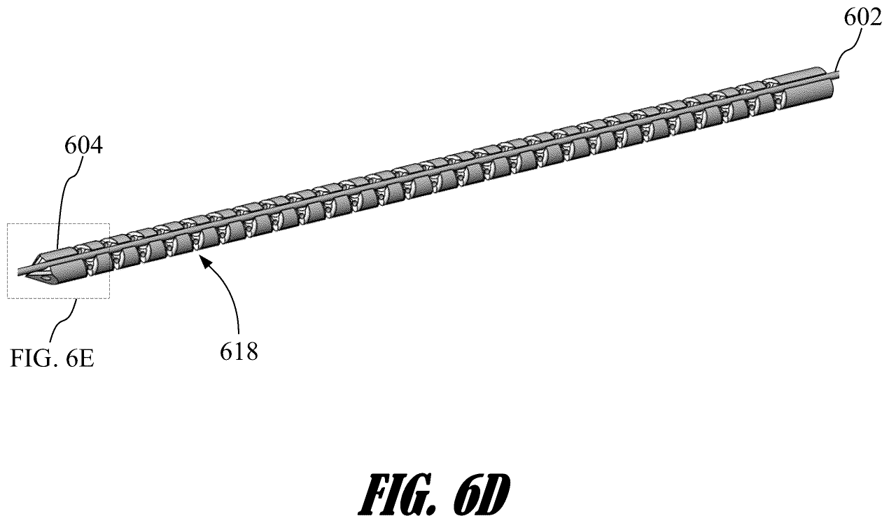

FIGS. 6A-6E illustrate another example of a sheath assembly 600 where the sheath 604 has relief areas. Similar to other embodiments, the sheath assembly 600 may include a sheath 604 that is configured to retain a tensioning element 602 within a core channel of the sheath 604. Further details regarding the features of the sheath assembly 600 may be seen in FIG. 6C. As illustrated, the sheath 604 may include sections 620. In some embodiments, the cross section of the sections 620 may be similar to the cross section of the sheath 304 illustrated in FIG. 3D.

Between the sections 620, relief areas 618 may be provided. Intermediate connections 619 may be provided in the relief areas 618 to connect adjacent sections 620 together. To the extent an operator desires to trim a portion of the sheath 604, the operator may trim the sheath at one of the intermediate connections 619. The intermediate connections 619 may generally possess a smaller thickness than the sections 620, making the intermediate connections 619 easier to trim. In some embodiments, the intermediate connections 619 may be manually trimmed at a desired point to trim the sheath 604, and this may be done by applying a sideloading force to the sheath 604 at the location where the breakage is desired. Trimming may be performed either before or after the sheath 604 have been placed inside the body of the patient, and trimming may be performed either before or after any tensioning element 602 has been added to the sheath 604. In the case where the tensioning element 602 has been assembled to the sheath 604 prior to trimming, excess sheath material may be trimmed and separated from the tensioning element 602 by simply sliding the excess sheath material off the tensioning element 602.

The sheath 604 may also include a tapered end 608. The tapered end 608 may aid the placement of the sheath 604 around a bone, reducing drag allowing the sheath 604 to more easily pass though or around cartilage, muscle, and other tissues covering or surrounding a bone.

Furthermore, the sheath 604 may include a bottom surface 613, and the bottom surface may be configured to contact a bone so that a tensioning element 602 placed in the core channel 610 (see FIG. 6E) of the sheath 604 may be directed away from the bone. The bottom surface 613 possesses a convex shape, but other shapes may be used. Furthermore, the intermediate connections 619 (see FIG. 6C) are provided at the bottom surface 613 of the sheath 604 to reduce the possibility that the tensioning element 602 will come in contact with the bone. However, the intermediate connections 619 may be provided elsewhere in other embodiments.

Furthermore, FIG. 6E illustrates a further enhanced perspective view of the sheath assembly 600 of FIG. 6D. In FIG. 6E, secondary channels 612 may be seen. In some embodiments, these secondary channels 612 may have a similar cross sectional shape as compared to the secondary channels 312 of FIG. 3C. Secondary channels 612 may be provided in each of the sections 620 in some embodiments.

While the sheath 504 and the sheath 604 illustrate example relief areas 518 and 618, relief areas may be provided in other ways. For example, relief areas may be provided in a honeycomb pattern, a square grid pattern, with round weepholes, or in an asymmetrical pattern.

Furthermore, the sheath may include a spike or spiked teeth to assist in restraining movement of the sheath relative to a bone. FIGS. 7A-7D illustrate an example sheath 704 having spiked teeth 728. FIG. 7A illustrates a side view of another example sheath assembly 700 having a tensioning element 702 and a sheath 704, and FIG. 7B-7D illustrate various enhanced views of the sheath assembly 700 of FIG. 7A where the spiked teeth 728 may be seen. The spiked teeth 728 may be directed into the bone or sternum, and this may assist in restraining movement without causing noticeable pain for the patient. Excess portions of the sheath 704 may be trimmed to reduce or eliminate the possibility of spiked teeth 728 coming into contact with other portions of the body. Furthermore, the spiked teeth 728 may possess a variety of shapes. For example, the spiked teeth 728 of FIGS. 7A-7D have the shape of a rectangular pyramid, but other shapes may be used.

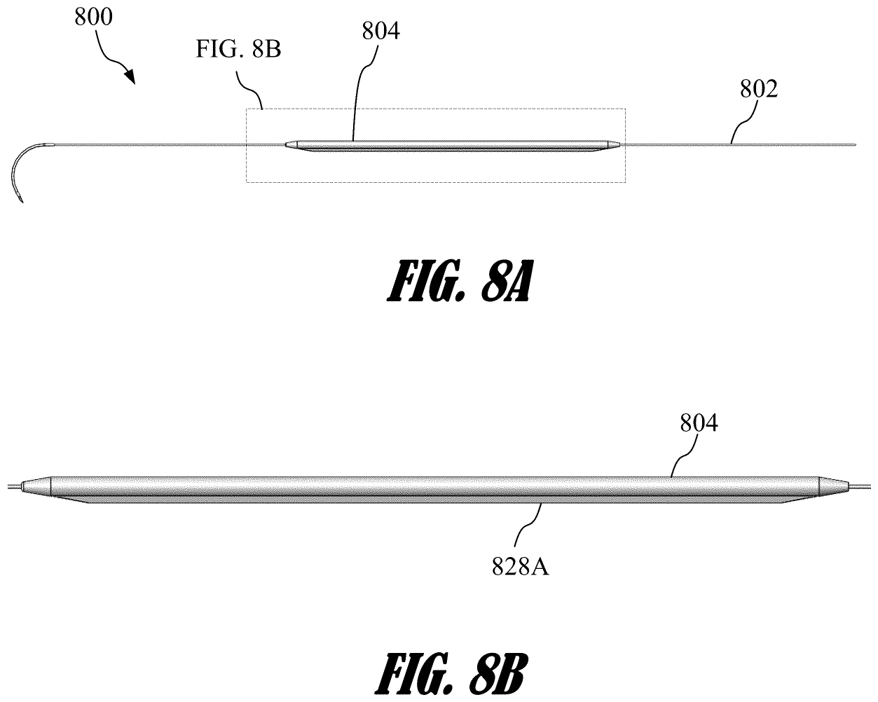

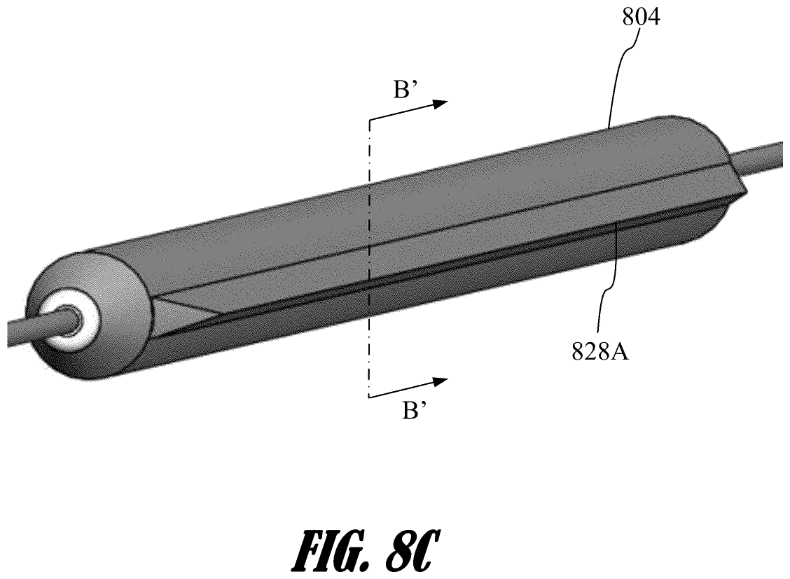

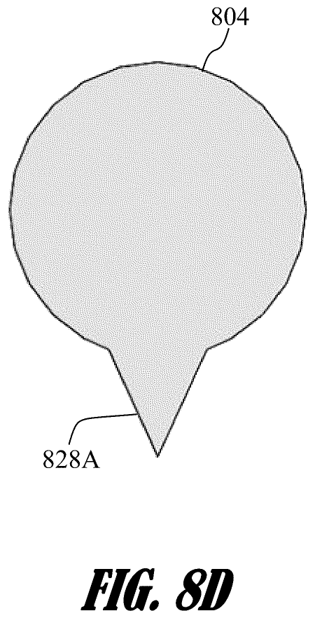

FIGS. 8A-8D illustrate another example sheath 804 having a single spike 828A running along the length of the sheath 804. FIG. 8A illustrates a side view of the sheath assembly 800 having a tensioning element 802 and a sheath 804. Additionally, FIG. 8B illustrates an enhanced side view of the sheath assembly 800 of FIG. 8A where the spike 828A may be seen, and FIG. 8C illustrates an enhanced perspective view of the sheath assembly 800 of FIG. 8A where the spike 828A may be seen. FIG. 8D illustrates a cross-sectional view of the sheath 804 of FIG. 8C about the line B′-B′.

The spike 828A may extend the entire length of the sheath 804 as illustrated in FIG. 8C. However, in other embodiments, multiple spikes may be provided in shorter segments, or a single spike may be provided in a shorter segment. While the spike 828A extends the entire length of the sheath 804, some portions of the sheath 804 may be provided without any spike 828A.

Spikes 828A of FIGS. 8A-8D and the spiked teeth 728 of FIGS. 7A-7D may have sharp or dull tips. Spikes 828A and spiked teeth 728 may be formed at the time of manufacturing for the sheath and may comprise the same material as the sheath. Alternatively, spikes 828A and spiked teeth 728 may be added to the sheath secondarily or may have a different material than the remainder of the sheath. The purpose of the spikes 828A and spiked teeth 728 may be to improve traction of the sheath against bone in some embodiments.

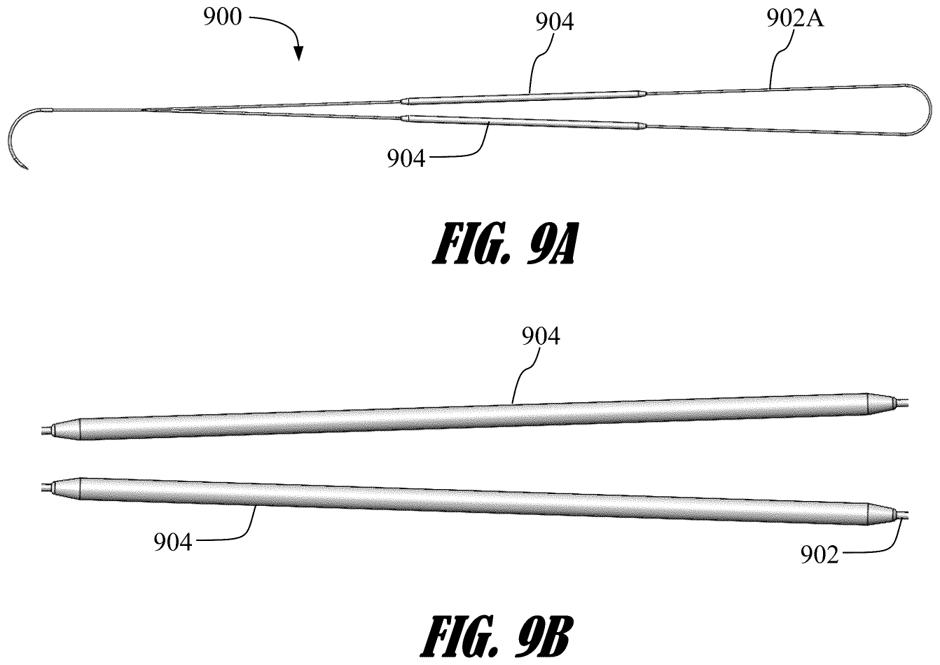

In some embodiments, multiple sheaths 904 may be used within a sheath assembly. FIG. 9A illustrates a side view of another example sheath assembly 900 having a double tensioning element 902A with sheaths 904 on the double tensioning element 902A, and FIG. 9B illustrates an enhanced side view of the sheath assembly 900 of FIG. 9A. As shown, multiple sheaths 904 are used in this embodiment, reducing the potential for contact between the double tensioning element 902 and any bone.

While various sheath assemblies and sheaths have been described herein, methods of using sheath assemblies and sheaths are also contemplated. FIG. 10 illustrates a method 1000 for creating and applying a sheath assembly. At operation 1002, a tensioning element is provided. This tensioning element may be a surgical wire. At operation 1004, a sheath is provided having a core channel. This sheath may be similar to any of the sheaths described above. At operation 1006, the tensioning element may be received in the core channel of the sheath.

At operation 1008, an unwanted portion of the sheath and/or the tensioning element may be trimmed. This may reduce the amount of excess material of the tensioning element and/or sheath material. At operation 1010, the tensioning element and sheath may be positioned around the bone. At operation 1012, the tensioning element and the sheath may be secured relative to the bone. Where the tensioning element is a wire, securing the tensioning element and the sheath relative to the bone may entail twisting the wire to reduce the size of the loop formed by the wire. Additionally or alternatively, securing the tensioning element and the sheath relative to the bone may entail applying the spikes or spiked teeth to the bone to gain traction between the bone and a surface of the sheath.

It should be understood that the operations discussed for the method 1000 may be performed in any order. Furthermore, some operations may be performed simultaneously with one another. Additionally, operations may be added or some operations may be removed. The method 1000 may be performed multiple times so that multiple sheath assemblies may be installed around a bone such as the sternum.

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the invention. In this regard, for example, different combinations of elements and/or functions than those explicitly described above are also contemplated within the scope of the invention. Although specific terms are employed herein, they are used in a generic and descriptive sense only and not for purposes of limitation.

That which is claimed:

1. A sheath comprising,
a core channel, the core channel configured to receive a tensioning element,
wherein the tensioning element is configured to circumscribe a bone, wherein the sheath is configured to be placed around a bone when the tensioning element received in the core channel circumscribes the bone, wherein the sheath is configured to prevent direct contact between the tensioning element and the bone as the tensioning element is tensioned.

2. The sheath of claim 1, wherein the sheath is configured to completely envelop the core channel.

3. The sheath of claim 1, wherein the sheath is configured to only partially envelop a portion of the core channel so that the core channel is partially exposed.

4. The sheath of claim 3, wherein the sheath has a non-uniform cross section, wherein the sheath has a buckle, wherein the buckle is configured to assist in guiding placement of the tensioning element in the core channel, wherein the buckle is configured to prevent inadvertent disengagement of the tensioning element from the sheath.

5. The sheath of claim 1, wherein the sheath has a tapered end, wherein the tapered end is configured to reduce resistance as the sheath is pulled through the body.

6. The sheath of claim 1, wherein the sheath has a non-uniform cross section.

7. The sheath of claim 6, wherein the sheath has at least one relief area having less material as compared to a remaining portion of the sheath, wherein the at least one relief area is configured to increase the ease of bending the sheath.

8. The sheath of claim 7, wherein the sheath contains at least one of a honeycomb pattern, a square grid pattern, round weepholes, or an asymmetrical pattern.

9. The sheath of claim 6, wherein the at least one relief area is configured to increase the ease of trimming the sheath to a desired length.

10. The sheath of claim 1, wherein the sheath has a non-uniform cross section, wherein the sheath has at least one relief area having less material as compared to a remaining portion of the sheath, wherein the at least one relief area is configured to permit free passage of blood and other biologic fluids to pass through to reach the fracture site and contribute to biological healing.

11. The sheath of claim 1, wherein the sheath includes malleable material, wherein the sheath is configured to conform to the contour of the bone when the sheath circumscribes the bone.

12. A sheath assembly comprising,
a tensioning element,
a sheath having a core channel, the core channel configured to receive the tensioning element,
wherein the sheath is configured to be placed around and positioned against a bone, wherein the sheath is configured to reduce direct contact between the tensioning element and the bone.

13. The sheath assembly of claim 12, wherein the tensioning element comprises at least one bead, wherein the at least one bead has an increased thickness compared to a remainder of the tensioning element, wherein the at least one bead is configured to restrain movement of the tensioning element relative to the sheath.

14. The sheath assembly of claim 12, wherein the tensioning element is a metal wire, wherein the tensioning element includes two free ends, wherein the tensioning element is configured to be tensioned by twisting the two free ends of the tensioning element together, wherein the tensioning element is configured to compress the sheath against the bone when the tensioning element is tensioned.

15. The sheath assembly of claim 14, wherein the sheath includes malleable material, wherein the sheath is configured to conform to the contour of the bone when the sheath circumscribes the bone.

16. The sheath assembly of claim 12, wherein the sheath has a non-uniform cross section.

17. The sheath assembly of claim 16, wherein the sheath has at least one relief area having less material as compared to a remaining portion of the sheath, wherein the at least one relief area is configured to increase the ease of bending the sheath.

18. The sheath assembly of claim 12, wherein the sheath has a non-uniform cross section, wherein the sheath has at least one relief area having less material as compared to a remaining portion of the sheath, wherein the at least one relief area is configured to permit free passage of blood and other biologic fluids to pass through to reach the fracture site and contribute to biological healing.

19. A method for applying a sheath assembly comprising:
providing a tensioning element;
providing a sheath having a core channel;
receiving the tensioning element in the core channel of the sheath;
positioning the tensioning element and the sheath around a bone; and
securing the tensioning element and the sheath relative to the bone.

20. The method of claim 19, wherein securing the tensioning element and the sheath relative to the bone includes applying one or more spikes to the bone.
